(19) United States
(12) Patent Application Publication
(10) Pub. No.: US 2019/0125869 A1
(43) Pub. Date: May 2, 2019

(54) PULSED ELECTROMAGNETIC THERAPY FOR DIRECTING AGENTS TO CANCER CELLS

(71) Applicant: MMTC, Inc., Princeton, NJ (US)

(72) Inventor: Fred Sterzer, Princeton, NJ (US)

(21) Appl. No.: 15/800,720

(22) Filed: Nov. 1, 2017

(51) Int. Cl. A61K 41/00; A61P 35/00; A61P 31/12; A61P 33/00; A61K 31/721; A61M 37/00; A61N 1/40; A61N 5/02; A61B 5/05; A61B 8/08

(52) U.S. Cl. CPC A61K 41/0028; A61P 35/00; A61P 31/12; A61P 33/00; A61K 31/721; A61M 2037/0007; A61N 1/40; A61N 5/022; A61B 5/0507; A61B 8/5261; A61M 37/0092

(57) ABSTRACT

Provided among other things is a method of treating a diseased tissue that is a cancer tissue, virally infected tissue, or a tissue infected with an obligate intracellular parasite, in a large animal comprising: (a) administering a therapeutic agent effective for treating the disease to the animal; and (2) using over a period of time one or more phased arrays of antennas to direct pulses of radio frequency or microwave radiation in permeability-enhancing amount to the affected tissue, wherein the pulsed radiation is effective to promote the activity of the therapeutic agent against cells of the cancerous tissue or against the activity of the pathogen.

The present application relates generally to methods of directing chemotherapy agents to malignant tissue, of cancer diagnostic methods, and of cancer imaging methods.

Some years ago a preliminary report of the scientific work that supports this application was presented orally at a conference. Copies of the presentation were not distributed. As reflected in a failure by the National Cancer Institute to fund further studies, the presentation was met with skepticism as to its broader implications. The oral presentation and the grant application were premised on the microwave radiation utilized in the preliminary work operating by causing electroporation through the membranes of the rat-hosted prostate cancer cells.

The NCI reviewers were apparently skeptical that the results implied a general strategy for treating cancer. That skepticism may have been based in part on the fact that the voltages induced by the microwave transmissions were too low to provoke electroporation. Thus, the positive results may have been seen as a special case stemming perhaps from the small size of the rat model, or other factors peculiar to the cancer model. Moreover, it may have been thought that the power needed to treat large animals would have led to thermal injury.

The pilot study, using 20 Copenhagen rats, demonstrated that microwave pulsing could sharply increase the uptake of systemically circulating fluorescin Dextran (10,000 daltons molecular weight) into implanted AT2 malignant prostate tumors. There was no significant uptake of Dextran by healthy cells that were correspondingly pulsed, indicating that a large therapeutic selectivity can be obtained by microwave pulsing. This pilot study further indicated that remission of tumors could be obtained by combining microwave pulsing and systemically administered Taxol.

It has now been concluded that the effects seen were not due to conventional electroporation, the process wherein electric fields induce the formation of holes in cell membranes. Applicant has deduced that the dramatic effect seen is due to dielectrophoresis, a process wherein a non-uniform electric field acts on a polarizable substrate.

In recent years important progress has been made in the application of electrophoresis to the separation in vitro of healthy and malignant cells, electrophoresis being the DC force that electrically polarizable materials such as cells experience when exposed to non-uniform electric fields [H. A. Pohl, “Deletrophoresis the behavior of neutral matter in nonuniform electric fields”, Cambridge University Press, 1978]. In particular it has been demonstrated that by means of electrophoresis it is possible to efficiently isolate in vitro everyone of the entire NCI-60 panel of circulating cancer cell types from normal blood cell types [Gascoyne et al., “Isolation of circulating tumor cells by dielectrophoresis”, Cancer (Basel), 6(1), 545-579, March 2014; Jubery et al., “Dielectrophoretic separation of bioparticles in microdevices: a review”, Electrophoresis 35(5) 691-713, March 2014; R. Pethic, “Review article—”, Dielectrophoresis: Status of the theory, technology, and applications”, Biomicrofluids 4(2) June 2010]. This separation effect is believed to be because of the structural differences between normal and malignant cells, malignant cells having typically 50% to 300% larger capacitance per unit area and also tend to have larger radii than their normal counter parts.

It has also been experimentally demonstrated that non-uniform electric fields at frequencies as high as microwaves will generate DC electrophoresis forces on polarizable materials [Watkins et al., “Measurement of microwave induced forces”, Cambridge Core, Volume 269, January 1992, 151]. This effect is important for in vivo medical applications of electrophoresis since radiofrequencies and microwaves can be noninvasively directed to locations inside the body by means of external antennas.

The structural differences between healthy and malignant cells that make possible the in vitro separation of malignant and healthy cells by electrophoresis also make it possible to selectively induce malignant cells in vivo to take up chemotherapeutic or diagnostic agents using high frequency pulsing. With electromagnetic pulses, for example, large cutaneous lesions, such as commonly occur in chest wall recurrence of breast cancer, can be pulsed with non-contacting applicators. Deep-seated lesions can be selectively targeted with radiofrequency pulses using multiple non-invasive antennas.

Now that the physical-chemistry basis of the observed selectivity has been deduced, a wide variety of therapeutic and diagnostic uses are implicated.

### SUMMARY

Provided for example is a method of treating a diseased tissue that is a cancer tissue, virally infected tissue, or a tissue infected with an obligate intracellular parasite, in a large animal comprising: (a) administering a therapeutic agent effective for treating the disease to the animal; and (b) using over a period of time one or more phased arrays of antennas to direct pulses of radio frequency or microwave radiation in permeability-enhancing amount to the affected tissue, wherein the pulsed radiation is effective to promote the activity of the therapeutic agent against cells of the cancerous tissue or against the activity of the pathogen.

Further provided for example is a method of treating a diseased tissue that is a cancer tissue, virally infected tissue, or a tissue infected with an obligate intracellular parasite, in a large animal comprising: (1) administering a therapeutic agent effective for treating the disease to the animal; and (2) using one or more antennas to direct pulses of radio frequency or microwave radiation in permeability-enhancing amount to a portion of the animal inclusive of diseased and substantial non-diseased tissue, wherein the pulsed microwaves are effective to promote the activity of the therapeutic agent against cells of the cancerous tissue or against the activity of the pathogen.

Additionally provided are methods of diagnosing or localizing diseased tissue, and method of treating such localized tissue.

### DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 depicts a controller for a radiometer; and

FIG. 2A is a shows a phased array system for transmitting electromagnetic radiation from a radiometer.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

Without being limited by theory, it is believed that cancer cells are selectively susceptible to pulsed electromagnetic radiation because of such factors as they are larger, have weaker membranes, and have different membrane compositions. Without being bound by theory, the same weaknesses are believed to be obtained from viral infection, from infection from obligate intracellular parasites including bacterial parasites such as bacteria of genii Chlamydia, Rickettsia, Coxiella, Mycobacterium (certain species such as Mycobacterium leprae and Mycobacterium tuberculosis) and the like. Accordingly, the treatments described here are believed to be applicable to such viral and obligate intracellular parasitic diseases. For viral diseases, the therapeutic agent can be any antiviral including without limitation Trifluridine, Vidarabine, Acyclovir, Ganciclovir, Penciclovir, Famciclovir, Ribavirin, Zidovudine, Amantadine, Rimantadine, Interferon α-2, Oseltamivir, Foscarnet, Oseltamivir, Fomivirsen, Zanamivir, Enfuvirtide, Azidothymidine, Efavirenz, Tenofovir, and the like. For antiparasite use, the therapeutic agent can be any antiparasitic including without limitation Bephenium, Diethylcarbamazine, Ivermectin, Niclosamide, Piperazine, Praziquantel, Pyrantel, Pyrvinium, Benzimidazoles, Albendazole, Flubendazole, Mebendazole, Thiabendazole, Benzyl benzoate, Benzyl benzoate/disulfiram, Lindane, Malathion, Permethrin, Benzyl alcohol, Piperonyl butoxide/pyrethrins, Spinosad, Crotamiton, and the like

Clinically significant in vivo dielectroporesis forces can be generated for example with low duty cycle, high-power radiofrequency or microwaves pulses. Using low duty cycle pulsing avoids excessive heating of the treated tissues, and the use of high peak power is can be useful because the time averaged DC force due to electrophoresis is proportional to the square of the applied electric field. High power radiofrequency and microwave pulses produce significant unidirectional stresses on cell membranes because of the large differences in the dielectric constants between the lipid membranes (low dielectric constant) and the interstitial and extrastitial fluids (high dielectric constant), as can very high field DC pulses [Hu et al., “Dielectrophoresis and electrorotation of spheroidal cells after nsPEF induced electroporation”, 4th International Conference on Bioinformatics and Biomedical Engineering, 18-20 Jun. 2010]. [The equations for the stresses that are produced by alternating electric fields at the interface between two bodies of different dielectric constants are given in most advanced textbooks on Electromagnetic Theory. See for example Chapter 2 of Stratton, “Electromagnetic Theory”, McGraw-Hill, 1941, incorporated by reference in its entirety]. High frequency pulses, unlike DC pulses, do not require implanted electrodes to produce in vivo dielectrophoresis because they can be broadcast into tissues by means of non-invasive antennas [applicators] and can penetrate tissues to great depth, their depth of penetration being a function of the frequency of the pulses.

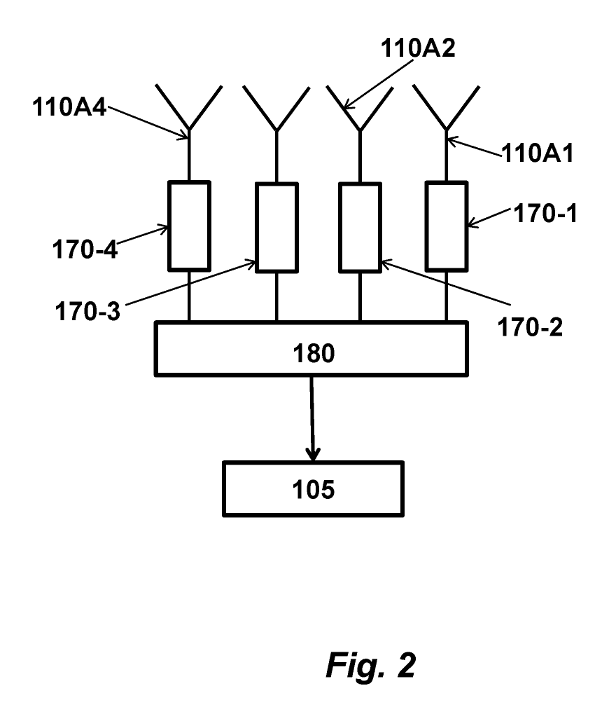

The structural differences between healthy and malignant cells that make possible the in vitro separation of malignant and healthy cells by electrophoresis also make it possible to selectively porate malignant cells in vivo using high frequency pulsing. With electromagnetic pulses, for example, large cutaneous lesions, such as commonly occur in chest wall recurrence of breast cancer, can be pulsed with non-contacting applicators. Deep-seated lesions can be selectively targeted with radiofrequency pulses using multiple non-invasive antennas as illustrated FIG. 2.

Microwaves have wavelengths ranging from one meter to one millimeter; with frequencies between 300 MHz and 300 GHz. For example, frequency bands of about 500 MHz can be used, such as for example from about 3.7 to about 4.2 GHz or about 1.2 to about 1.7 GHz. For this poration use, it is believed that microwave and radio frequencies can be used (e.g., about 1 MHz to about 300 MHz). In embodiments, frequencies from about 1 MHz to about 300 GHz can be used.

A phased array of antennas can be utilized to concentrate the pulsed electromagnetic energy at the target lesion area, even one deep in the subjects tissue. In the mode of assisting chemotherapy, targeting can encompass more than the lesion, as the pulsed radiation is not effective to increase the permeability of the chemotherapeutic agent into adjacent normal tissue.

For this phased array, for example, several small printed circuit transmitting antennas (e.g., printed circuit X-slot micro-strip antennas) can be connected in parallel and placed around the surface location corresponding to the internal lesion that is to be treated. (These can also be used in the invention as equivalent to one large antenna but more easily placed in contact with the skin.) The array can be operated as a radiometer in receiving mode to monitor the temperature of the target tissue. If temperature rise is more than the target parameters, the duty cycle for pulsing the radiometer can be adjusted. A feedback circuit from the radiometer, for example via a controller, assures the measured tumor temperatures are kept under to a preset value. A phase shifter (line stretcher) after each antenna can be used to obtain the phase shift. One can adjust the phase shifters behind the radiometer antennas to obtain a maximum temperature reading of the tumor. This read-mode procedure can optimize directing the pulsed electromagnetic power to a metabolically active malignant tumor whose temperature is elevated.

For example, as shown in FIG. 2, Antennas 110A1 to 100A4 can be used in place of antenna 110A. Adjustable line stretchers 170-1 to 170-4 can be used to establish the phase separation. Combiner 180 is used to provide appropriate connection to the radiometer 105.

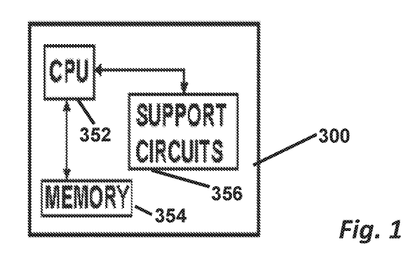

The radiometer and/or exterior elements case can incorporate electronic controllers, such as controller 300 (FIG. 1). The controller 300 comprises a central processing unit (CPU) 354, a memory 352, and support circuits 356 for the CPU 354 and is coupled to and controls one or more of the various elements of the radiometer or, alternatively, via computers (or controllers) associated with radiometer. The controller 300 may be one of any form of general-purpose computer processor that can be used for controlling various devices and sub-processors. The memory, or computer-readable medium, 352 of the CPU 354 may be one or more of readily available memory such as random access memory (RAM), read only memory (ROM), flash memory, floppy disk, hard disk, or any other form of digital storage, local or remote. The support circuits 356 are coupled to the CPU 354 for supporting the processor in a conventional manner. These circuits can include cache, power supplies, clock circuits, input/output circuitry and subsystems, and the like. Methods of operating the TNI device 100 may be stored in the memory 352 as software routine that may be executed or invoked to control the operation of the mobile radiometer, such as activating the power consuming components, and the like. Software routines may also be stored and/or executed by a second CPU (not shown) that is remotely located from the hardware being controlled by the CPU 354.

The successful experiments described below that were performed on the rats were against a particular type of implanted prostate tumor, were at a particular frequency, a particular power level, and used a particular chemotherapeutic agents (taxol). For different types of human malign tumors, located at different distances from the surface skin of the patient, it is anticipated that different power levels, different frequencies, or different chemotherapeutic agents, can be more effective. Relative effectiveness, given different values of frequency, power level, or agent for a particular clinical use can be determined with experiments such as those outlined below.

### Ultrasound

In all embodiments, ultrasound waves can be focused on the tissue in question to accentuate the effects of the pulsed electromagnetic radiation. Ultrasound pulses can be time coherent with microwave or RF pulses, i.e. make ultrasound pulses at the same time or just before electromagnetic pulses. Dielectrophoresic forces are created by non-uniform electric fields, and the electric fields acting on cell membranes whose shapes are distorted by the ultrasound pulses are more non-uniform than those on cell membranes not exposed to ultrasound. One can focus several distinct ultrasound beams on deep-seated tissues to accentuate permeability.

DC forces due to electrophoresis on a particle made of electrically polarizable material such as a cell occur if the particle is exposed to non-uniform electric fields. If the shape of the particle (cell) is distorted by the mechanical forces due to ultrasound it becomes more likely that different parts of the particle will be exposed to slightly different electric fields.

### Normal Cells

While as discussed above, diseased cells can be more susceptible to the methods of the invention, yet some effect on reagent permeability should be realized by normal cells.

For example, an area of the brain may be treated with the radiation (and optionally ultrasound) in conjunction with a drug for a neurological disorder. The pulsed radiation increasing flow of the drug across the blood-brain barrier. For example, drugs targeting Alzheimer's disease can be administered in this fashion.

### Identifying Aggressive Malignancies

With the invention, one can distinguish fast growing malignant cells from slow growing by determining how much peak pulse power is needed to get the chemotherapeutic agent into the cells (which can be modeled with tracer agents). The lower the peak power, the more aggressive are the tumor cells. (This is indicated to the inventors by experiments on isolating transformed cells. Gascoyne and Shim, Cancers 2014, 6(1), 545-579 (Isolation of Circulating Tumor Cells by Dielectrophoresis).)

### Additional Function

Frequencies in the range of 100-300 kHz with intensities of 1-3 volt/cm are currently used in FDA approved cancer therapies referred to as ‘Tumor treating fields (TTF fields)” to prevent the division and therefore the proliferation of cancer cells [A. M. Davies, U. Weinberg, Y. Palti, “Tumor treating fields: a new frontier in cancer therapy”, Ann NY Academia Science, July 2013. Many more references on TTF fields can be found in Wikipedia. The exact optimum frequency to use to inhibit cell division in a particular cancer is experimentally determined]. Instead of using low frequency electric fields, it would be advantageous in most instances to use radiofrequencies (e.g. about 1 MHz or above) or microwaves that are modulated at the appropriate kHz frequencies to generate the dielectrophoresic forces that prevent cancer cell division. (When about to divide cells assume an hourglass shape. Any applied RF or microwave power applied to cells about to undergo division will be concentrated in the narrow parts of the hour glass shaped dividing cells, generating dielectrophoresic forces that when large enough can prevent the cells from dividing.) The advantages of using modulated RF and microwaves instead of the low frequencies TTF fields is that modulated RF and microwaves, unlike TTF fields, can be noninvasively directed by using multiple antennas to deep-seated malignant tumors, and could therefore be used to produce cancer cell apoptosis in all types of malignancies

### Misc.

“Imaging agents” are agents that can be localized in an “imaging tool,” such as a nuclear imaging device (such as positron emission tomography device), a magnetic resonance imaging device, an x-ray device, a computed tomography device, or the like. Examples for nuclear imaging include for example reagents including technetium (⁹⁹Tc), gallium (⁶⁷Ga), thallium (e.g., ²⁰⁴Tl), ⁶⁴Cu, ¹⁸F and the like. Examples for MRI include for example gadolinium, reagents including gadolinium and the like. The reagents can be formed for example by compounds that coordinate imaging components (e.g., metals) or otherwise incorporate the imaging components. Preferably, imaging reagents are sized to resist diffusion into cells.

In many embodiments, the subjects for treatment are large animals (patients), in that they are at least about 10-fold greater in mass than 90 day old Copenhagen rats. As such, the treatment animals are generally about 10 kg or greater in mass.

“Substantial” tissue adjacent to removed tissue or substantial non-diseased tissue is tissue in the amount of 40% of the mass of the removed or diseased tissue, or more.

To treat indications with a therapeutic agent, an “effective amount” of a therapeutic agent will be recognized by clinicians but includes an amount effective to treat, reduce, alleviate, ameliorate, eliminate or prevent one or more symptoms of the condition sought to be treated, or alternately, the condition sought to be avoided, or to otherwise produce a clinically recognizable favorable change in the condition or its effects.

A “target AUC” for a therapeutic agent is the lowest AUC deemed effective for the therapeutic agent relative to the disease, with the amount determined in the absence of the pulsed electromagnetic radiation of the invention.

Specific embodiments according to the methods of the present invention will now be described in the following examples. The examples are illustrative only, and are not intended to limit the remainder of the disclosure in any way.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

A laminate is a bonding, fusing, adhesion, or the like between polymer layers, or between polymer and fabric layers, such that in the range of anticipated use the laminate is a unitary structure.

Where a sentence states that its subject is found in embodiments, or in certain embodiments, or in the like, it is applicable to any embodiment in which the subject matter can be logically applied.

### Example 1

The experiments reported at this meeting used 25 three-month old male Copenhagen rats. The tumor model was AT2 prostatic tumor of rats obtained from the John Hopkins Medical Center. The natural course of this tumor simulates that that of human prostatic cancer. The lower chest/abdomen of the rats were implanted with 3 cubic millimeters of tumor tissue. Experiments were started when the implanted tumors grew to about 10 mm. The microwave generator used in the experiments was a magnetron oscillator with following characteristics: Frequency=2.82 GHz, Peak power=166 kW, Pulse width=0.25 microseconds, Average power=0.55 watt. Treatment times were 30 minutes. The microwave power from the magnetron oscillator was fed to the surface of tissues of by a dielectric antenna applicator. The temperatures of the tumors or healthy tissues exposed to microwave pulsing increased by only one or two degree Celsius during the pulsing

14 anesthetized rats with contralateral tumors were injected with fluorescin isothiocyanate Dextran with molecular weight 12,000 Daltons [Sigma-Aldrich St. Louis Mo., USA] 20 mg in 0.5 ml of saline into the tail vein. In each case one of the tumors was pulsed with microwaves, while the contralateral tumor was not pulsed.

There was significant uptake of Dextran by the cells of the tumors subjected to pulsing, while there was almost no uptake of non-pulsed tumors. This was shown with a phase microscope that showed cell boundaries and with the same field of the phase microscope using ultraviolet light to indicate where Dextran had penetrated the cells.

### Example 2

The comparable experiment to Example 1 was conducted with normal muscle tissue. There was no significant uptake of Fluorescine Dextran by the muscle cells exposed to pulsing.

### Example 3

One rat with four implanted tumors, two in each flank, was used. Taxol was injected intravenously at 5 mg/kg. The two lower (right and left) tumors were treated as described above with pulsed microwaves, beginning immediately after the injection. Tumor growth was monitored over 14 days.

The results were that the control tumors remained large, and the two treated tumors had almost disappeared at 5 days. After 14 days, the treated tumors were barely detectable, and the control tumors continued to grow.

This invention described herein is of a enhanced chemotherapy, diagnostic methods and related subject matter. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims. More specifically, those of skill will recognize that any embodiment described herein that those of skill would recognize could advantageously have a sub-feature of another embodiment, is described as having that subfeature.

The method can be further described with reference to the following numbered embodiments:

### Embodiment A1

A method of treating a diseased tissue that is a cancer tissue, virally infected tissue, or a tissue infected with an obligate intracellular parasite, in a large animal comprising: (a) administering a therapeutic agent effective for treating the disease to the animal; and (b) using over a period of time one or more phased arrays of antennas to direct pulses of radio frequency or microwave radiation in permeability-enhancing amount to the affected tissue, wherein the pulsed radiation is effective to promote the activity of the therapeutic agent against cells of the cancerous tissue or against the activity of the pathogen.

### Embodiment A2

The method of an A Embodiment, wherein the large animal is a human.

### Embodiment A3

The method of an A Embodiment, wherein the phased array is operated over times and locations so as to apply the permeability-enhancing amount of radiation to a portion of the animal inclusive of diseased and substantial non-diseased tissue.

### Embodiment A4

The method of an A Embodiment, wherein the plasma Area Under the Curve for the therapeutic agent for the period of time is 75% or less than the target AUC for the therapeutic agent normalized to the same period of time.

### Embodiment A5

The method of an A Embodiment, comprising the step of, previous to the using the phased arrays, surgically excising acutely disease-affected tissue, and treating as the affected tissue that tissue adjacent the excised tissue or tissue susceptible to metastasis or otherwise acquiring the disease state from acutely disease-affected tissue.

### Embodiment A6

The method of an A Embodiment, further comprising applying ultrasound to the affected tissue in conjunction with the radio frequency or microwave radiation.

### Embodiment B1

A method of treating a diseased tissue that is a cancer tissue, virally infected tissue, or a tissue infected with an obligate intracellular parasite, in a large animal comprising: (A) administering a therapeutic agent effective for treating the disease to the animal; and (B) using one or more antennas to direct pulses of radio frequency or microwave radiation in permeability-enhancing amount to a portion of the animal inclusive of diseased and substantial non-diseased tissue, wherein the pulsed microwaves are effective to promote the activity of the therapeutic agent against cells of the cancerous tissue or against the activity of the pathogen.

### Embodiment B1

The method of a B Embodiment, wherein the large animal is a human.

### Embodiment B2

The method of a B Embodiment, wherein the plasma Area Under the Curve for the therapeutic agent for the period of time is 75% or less than the target AUC for the therapeutic agent normalized to the same period of time.

### Embodiment B3

The method of a B Embodiment, comprising the step of, previous to the using the antennas, surgically excising acutely disease-affected tissue, and treating as the affected tissue that tissue adjacent the excised tissue or tissue susceptible to metastasis or otherwise acquiring the disease state from acutely disease-affected tissue.

### Embodiment B4

The method of a B Embodiment, further comprising applying ultrasound to the diseased and substantial non-diseased tissue in conjunction with the radio frequency or microwave radiation.

### Embodiment C1

A method of diagnosing disease or localizing cells or tissue that is diseased in that it is cancerous, virally infected, or a infected with an obligate intracellular parasite, the method comprising: (I) administering an imaging agent to the animal; (II) using one or more antennas to direct pulses of radio frequency or microwave radiation in permeability-enhancing amount to a substantial portion of the animal inclusive of diseased and substantial non-diseased tissue, wherein the pulsed microwaves are effective to promote the entry of the imaging agent into the diseased cell to tissue; and (Ill) after directing the microwave radiation, using an imaging tool to locate the imaging agent in the animal.

### Embodiment C2

The method of a C Embodiment, wherein the imaging tool is separately used (a) after administering and before directing radio frequency or microwave radiation and (b) after directing radio frequency or microwave radiation, providing comparative results showing tissue in which uptake of the imaging agent was enhanced by the radiation.

### Embodiment C3

The method of a C Embodiment, further comprising applying ultrasound to the substantial portion in conjunction with the radio frequency or microwave radiation.

### Embodiment C-A1

A method of treating a diseased tissue, comprising localizing the tissue pursuant to a C Embodiment, and: (1) thereafter administering a therapeutic agent effective for treating the disease to the animal; and (2) using one or more antennas to direct pulses of radio frequency or microwave radiation in permeability-enhancing amount to a so localized portion of the animal, wherein the pulsed radiation is effective to promote the activity of the therapeutic agent against cells of the cancerous tissue or against the activity of the pathogen.

### Embodiment C-A2

The method of a C-A Embodiment, wherein the plasma Area Under the Curve for the therapeutic agent for the period of time is 75% or less than the target AUC for the therapeutic agent normalized to the same period of time.

### Embodiment C-A3

The method of a C-A Embodiment, further comprising applying ultrasound to the localized portion of the animal in conjunction with the radio frequency or microwave radiation.

### Embodiment C-B1

A method of treating a diseased tissue, comprising localizing the tissue pursuant to a C Embodiment, and: (1) thereafter utilizing the localization to surgically remove diseased tissue; (2) administering a therapeutic agent effective for treating the disease to the animal; and (3) using one or more antennas to direct pulses of radio frequency or microwave radiation in permeability-enhancing amount to a so localized portion of the animal, inclusive of substantial tissue adjacent to the removed tissue, wherein the pulsed radiation is effective to promote the activity of the therapeutic agent against cells of the cancerous tissue or against the activity of the pathogen.

### Embodiment C-B2

The method of a C-B Embodiment, wherein the plasma Area Under the Curve for the therapeutic agent for the period of time is 75% or less than the target AUC for the therapeutic agent normalized to the same period of time.

### Embodiment C-B3

The method of a C-B Embodiment, further comprising applying ultrasound to the localized portion of the animal in conjunction with the radio frequency or microwave radiation.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A method of treating a diseased tissue that is a cancer tissue, virally infected tissue, or a tissue infected with an obligate intracellular parasite, in a large animal comprising:
administering a therapeutic agent effective for treating the disease to the animal; and
using over a period of time one or more phased arrays of antennas to direct pulses of radio frequency or microwave radiation in permeability-enhancing amount to the affected tissue, wherein the pulsed radiation is effective to promote the activity of the therapeutic agent against cells of the cancerous tissue or against the activity of the pathogen.

2. The method of claim 1, wherein the large animal is a human.

3. The method of claim 1, wherein the phased array is operated over times and locations so as to apply the permeability-enhancing amount of radiation to a portion of the animal inclusive of diseased and substantial non-diseased tissue.

4. The method of claim 1, wherein the plasma Area Under the Curve for the therapeutic agent for the period of time is 75% or less than the target AUC for the therapeutic agent normalized to the same period of time.

5. The method of claim 1, comprising the step of, previous to the using the phased arrays, surgically excising acutely disease-affected tissue, and treating as the affected tissue that tissue adjacent the excised tissue or tissue susceptible to metastasis or otherwise acquiring the disease state from acutely disease-affected tissue.

6. The method of claim 1, further comprising applying ultrasound to the affected tissue in conjunction with the radio frequency or microwave radiation.

7. A method of treating a diseased tissue that is a cancer tissue, virally infected tissue, or a tissue infected with an obligate intracellular parasite, in a large animal comprising:
administering a therapeutic agent effective for treating the disease to the animal; and
using one or more antennas to direct pulses of radio frequency or microwave radiation in permeability-enhancing amount to a portion of the animal inclusive of diseased and substantial non-diseased tissue, wherein the pulsed microwaves are effective to promote the activity of the therapeutic agent against cells of the cancerous tissue or against the activity of the pathogen.

8. The method of claim 7, wherein the large animal is a human.

9. The method of claim 7, wherein the plasma Area Under the Curve for the therapeutic agent for the period of time is 75% or less than the target AUC for the therapeutic agent normalized to the same period of time.

10. The method of claim 7, comprising the step of, previous to the using the antennas, surgically excising acutely disease-affected tissue, and treating as the affected tissue that tissue adjacent the excised tissue or tissue susceptible to metastasis or otherwise acquiring the disease state from acutely disease-affected tissue.

11. The method of claim 7, further comprising applying ultrasound to the diseased and substantial non-diseased tissue in conjunction with the radio frequency or microwave radiation.

12. A method of diagnosing disease or localizing cells or tissue that is diseased in that it is cancerous, virally infected, or a infected with an obligate intracellular parasite, the method comprising:
administering an imaging agent to the animal;
using one or more antennas to direct pulses of radio frequency or microwave radiation in permeability-enhancing amount to a substantial portion of the animal inclusive of diseased and substantial non-diseased tissue, wherein the pulsed microwaves are effective to promote the entry of the imaging agent into the diseased cell to tissue; and
after directing the microwave radiation, using an imaging tool to locate the imaging agent in the animal.

13. The method of claim 12, wherein the imaging tool is separately used (a) after administering and before directing radio frequency or microwave radiation and (b) after directing radio frequency or microwave radiation, providing comparative results showing tissue in which uptake of the imaging agent was enhanced by the radiation.

14. The method of claim 12, further comprising applying ultrasound to the substantial portion in conjunction with the radio frequency or microwave radiation.

15. A method of treating a diseased tissue, comprising localizing the tissue pursuant to claim 12, and:
thereafter administering a therapeutic agent effective for treating the disease to the animal; and
using one or more antennas to direct pulses of radio frequency or microwave radiation in permeability-enhancing amount to a so localized portion of the animal, wherein the pulsed radiation is effective to promote the activity of the therapeutic agent against cells of the cancerous tissue or against the activity of the pathogen.

16. The method of claim 15, wherein the plasma Area Under the Curve for the therapeutic agent for the period of time is 75% or less than the target AUC for the therapeutic agent normalized to the same period of time.

17. The method of claim 15, further comprising applying ultrasound to the localized portion of the animal in conjunction with the radio frequency or microwave radiation.

18. A method of treating a diseased tissue, comprising localizing the tissue pursuant to claim 12, and:
thereafter utilizing the localization to surgically remove diseased tissue;
administering a therapeutic agent effective for treating the disease to the animal; and
using one or more antennas to direct pulses of radio frequency or microwave radiation in permeability-enhancing amount to a so localized portion of the animal, inclusive of substantial tissue adjacent to the removed tissue, wherein the pulsed radiation is effective to promote the activity of the therapeutic agent against cells of the cancerous tissue or against the activity of the pathogen.

19. The method of claim 18, wherein the plasma Area Under the Curve for the therapeutic agent for the period of time is 75% or less than the target AUC for the therapeutic agent normalized to the same period of time.

20. The method of claim 18, further comprising applying ultrasound to the localized portion of the animal in conjunction with the radio frequency or microwave radiation.
